(19) United States
(12) Patent Application Publication
(10) Pub. No.: US 2004/0064118 A1
(43) Pub. Date: Apr. 1, 2004

(54) Absorbent product

(76) Inventor: Francisco J. V. Hernandez, Sao Jose dos Campos (BR)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/623,879

(22) Filed: Jul. 18, 2003

(30) Foreign Application Priority Data
Jul. 19, 2002 (BR) PI 0202912-0

(51) Int. Cl. A61F 13/15; A61F 13/20

(52) U.S. Cl. 604/367

(57) ABSTRACT

An absorbent product, particularly for the absorption of body exudates, comprising an inner sheet (2) to be positioned close to the wearer's body, an outer sheet (4) to be positioned close to the wearer's garments and an absorbent core (3) positioned between said outer and inner faces (2, 4), the absorbent core (3) being enveloped by a transfer layer (5), made of an absorbent material, which comprises a polymeric material with a basis weight substantially between 8 and 20 g/m², and, additionally, at least one permanent surfactant element. The product (1) of the present invention presents, as advantages, a higher absorption capacity and a lower liquid penetration time, even after successive discharges, continuing to rapidly absorb and conduct the body fluids from the discharge region to the absorbent core (3), allowing the correct storage thereof. Also, the transfer layer (5) offers high mechanical strength, reducing the occurrence of structural collapse of the absorbent core (3) due to moisture combined with the application of forces. Furthermore, all these advantages are achieved, while maintaining a reduced manufacturing cost for the absorbent product (1), thus maximizing its potential of penetrating in the consumer market.

### FIELD OF THE INVENTION

The present invention refers to an absorbent product particularly for the absorption of body exudates, which presents properties such as liquid penetration time, impact absorption capacity, and integrity of the absorbent core, which are significantly improved in relation to those of the existing products.

### DESCRIPTION OF THE PRIOR ART

Conventional absorbent products, such as sanitary napkins, disposable diapers for babies, incontinence products and the like, are provided with an absorbent core blended with an absorbent or even super-absorbent material, which is optionally enveloped by a layer made of a material presenting fast liquid penetration time which is capable of rapidly transporting the body exudates to the absorbent material, aiming at the maximum possible retention.

However, when these products are in use, they may be submitted to several stresses of mechanical nature, such as twists and compressions (for example, the compression made by the legs of a sanitary napkin wearer), as well as in relation to the quantity of liquid to be absorbed. As it is well known, the quantity of body exudates eliminated by a wearer (urine, menses, various discharges, etc.) can greatly vary, as well as the time interval in which these discharges occur. Particularly in the case of successive discharges, the possibility of leakage of exudates increases considerably, since there is not sufficient time between the discharges for the liquid to be transported from the discharge area to the absorbent core areas with higher absorption capacity.

As a general rule, as the absorbent product becomes saturated, its absorption capacity is gradually reduced and its penetration time increases, making the absorbent product more inclined to leakage.

For better describing the construction of these absorbent products, they comprise a liquid permeable inner sheet (which contacts the wearer's skin), an inner absorbent core, which is the main component responsible for the absorption and retention of body exudates, and an impermeable outer sheet (which contacts the wearer's undergarment). As already mentioned, some absorbent products use a layer made of a material presenting high fluid absorption and good liquid penetration time, as a kind of coating for the absorbent core. Preferably a paper of the tissue type is used. This tissue paper could, alternatively, not only envelop the absorbent core but also be provided in the part of the latter facing the wearer's body or in its part facing the opposite direction. This component may also be called a transfer layer, to be described below. The transfer layer has the primary function of helping to transfer body exudates from the discharge region to the absorbent core.

The transfer layer has the important mission to “conduct” body exudates from the discharge region as rapidly and efficiently as possible, which is achieved by the phenomenon of capillarity. Another function of this layer, particularly when it envelops the absorbent core, is to assure the integrity of said core, especially when it is exposed to a high quantity of body exudates, which may reduce the mechanical strength of said core.

Moreover, a reduction in the absorption capacity and an increase in the penetration time may occur due to the removal of moistening agent (surfactant) from the permeable inner sheet layer after successive liquid discharges. The integrity of the core is also a factor that influences the absorption properties of the disposable products. Excess humidity may reduce the stability of both the tissue paper and the absorbent core, and it may cause a structural collapse even under a small mechanical stress, such as the compression caused by the wearer's legs, in cases where the absorbent product is a sanitary absorbent or a diaper.

With the objective of improving the penetration time of fluid into the absorbent core, transfer layers have been increasingly incorporated in absorbent structures between the liquid permeable inner layer and the absorbent core. In order to improve the performance of the absorbent product in relation to those products containing tissue paper, webs made of high denier synthetic fibers have been used, such as polypropylene, polyester and the like, which are agglutinated and thermoformed, so as to produce a layer of high basis weight, low density and high porosity which allows a rapid liquid penetration. This solution generally achieves good results, however due to the high cost of the materials used and to modifications in the production process required for its application, the problem is not solved at a cost compatible with the products of low price and high market penetration, particularly in those countries where a majority of the population have low incomes.

### OBJECTIVES OF THE INVENTION

The objective of the present invention is to provide an absorbent product, particularly for the absorption of body exudates, which presents a transfer layer having good properties of liquid transport and mechanical strength even when humid, thus contributing to the efficient transfer of exudates from the discharge region to the absorbent core, and contributing to the maintenance of the physical integrity of said core, avoiding its structural collapse when submitted to certain mechanical stresses.

Obviously, the manufacturing cost of the absorbent product thus configured should be substantially reduced in relation to the already developed alternatives mentioned above.

### BRIEF DESCRIPTIONS OF THE INVENTION

In the present invention, the improvement in the absorption capacity and penetration time of the transfer layer to absorb and conduct body exudates to an absorbent core, even after successive discharges, is achieved by enveloping the absorbent core with a transfer layer made with a polymeric material and at least one permanent surfactant and having a low basis weight (preferably in the range of 8-12 g/m²). As used herein, “permanent surfactant” means a surfactant that enables a polymeric material to which it is applied to remain wettable by aqueous body fluids, or maintain its hydrophilicity, even after repeated insults of aqueous body fluids. In other words, a permanent surfactant does not “wash off” the polymeric material to which it is applied under normal conditions of use in which multiple insults of fluids are applied to the polymeric material. Preferably the transfer layer is a non-woven web of 100% polypropylene fibers with low Denier (1.7D) obtained by the spunbonding manufacturing process (formation via continuous spinning). In order to achieve the desired results, it is necessary that the non-woven web be treated with a “permanent surfactant” to allow the material to maintain its properties of rapid absorption and rapid incorporation of liquids into the absorbent core. A preferred permanent surfactant is LERTISAN HD-14/HD-20, which is available from Zschimmer & Schwarz GmbH & Co. of Lahnstein, Germany. Likewise, the inner sheet (facing the wearer's body), which receives the liquid discharges, should be treated with this “permanent surfactant”, in order to form a layer system for the rapid penetration of body exudates.

Thus, the objectives of the present invention are achieved by an absorbent product, particularly for the absorption of body exudates, comprising an inner sheet to be positioned close to the wearer's body, an outer sheet to be positioned close to the wearer's undergarment, and an absorbent core positioned between said inner and outer sheets, the core being enveloped by a transfer layer made of absorbent material, the absorbent material layer comprising a polymeric material and presenting a basis weight substantially between 8 and 20 g/m², and further comprising at least one permanent surfactant element.

The present invention presents, as advantages, a higher absorption capacity and a lower penetration time, even after successive fluid discharges, continuing to rapidly absorb and conduct the body fluids from the discharge region to the absorbent core, making possible the correct storage of said fluids. Furthermore, the absorption time is even shorter than other products, enhancing these advantages.

Additionally, the transfer layer thus configured offers high mechanical strength, which reduces the occurrence of structural collapse of the absorbent core due to moisture, combined with the application of forces.

Moreover, all these advantages are achieved, while maintaining a reduced manufacturing cost for the absorbent product, thus maximizing its potential of penetrating in the consumer market.

### BRIEF DESCRIPTION OF THE DRAWINGS

The present invention will be described below in more details, based on an exemplary embodiment shown in the drawings, in which:

FIG. 1 is a top plan schematic view of an embodiment of the absorbent product of the present invention;

FIG. 2 is a sectional schematic view taken along line A-A of the absorbent product of the present invention illustrated in FIG. 1;

FIG. 3 is a first graph of penetration time of multiple insults of fluid on embodiments of the present invention and on comparative examples;

FIG. 4 is a second graph penetration time of multiple insults of fluid on embodiments of the present invention and on comparative examples; and

FIG. 5 is a third graph penetration time of multiple insults of fluid on embodiments of the present invention and on comparative examples.

### DETAILED DESCRIPTION OF THE INVENTION

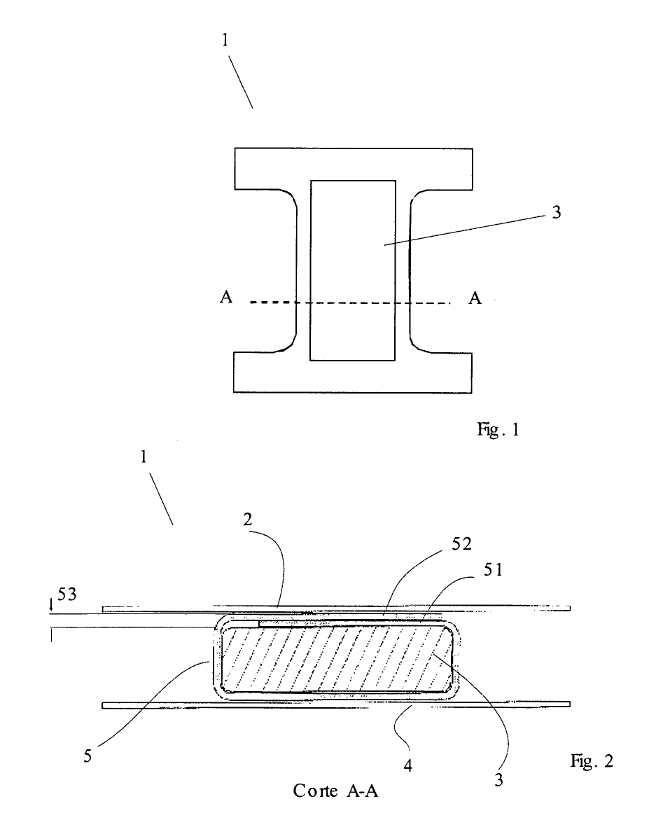

According to a preferred embodiment and as seen in FIGS. 1 and 2, the absorbent product 1 of the present invention (which can be a sanitary napkin, a disposable diaper for babies, an incontinence product, or the like), comprises a first inner sheet 2 to be positioned close to the wearer's body (i.e., facing the wearer's body when the absorbent product 1 is used), and an outer sheet 4 to be positioned close to the wearer's garments (e.g., facing the wearer's undergarment when the absorbent product 1 is used). Both the inner and outer sheets 2, 4, are substantially parallel to each other.

In order to allow the correct operation of the invention, the inner sheet 2 is comprised of a substantially liquid permeable material, whereas the outer sheet 4 is comprised of a substantially liquid impermeable material. This construction is necessary, since the inner sheet 2 allows the passage and absorption of the body fluid to the absorbent core, whereas the outer sheet 4 prevents this fluid from reaching the wearer's undergarment, which could cause extreme discomfort of use.

Preferably, the inner sheet 2 comprises a non-woven permeable web composed of 100% polypropylene or the like, with a preferred basis weight substantially between 8 and 20 g/m², and obtained by a spunbonding manufacturing process (formation via continuous spinning). In order to guarantee its fluid absorption and transport properties, the inner sheet 2 is treated with a permanent surfactant of the LERTISAN HD-20 type in quantities of 0.5-1.2%. However, it is possible to use other surfactant elements.

The absorbent product 1 further contains an absorbent core 3 positioned between said inner sheet 2 and outer sheet 4. The absorbent core 3, which is positioned such as mentioned above, comprises a structure preferably formed by a cellulosic material with high capacity of absorbing body exudates, such as for example, cellulose pulp. In order to improve the liquid storage properties thereof, it is possible to optionally add superabsorbent polymers (SAP), which allow the absorption and retention of a much higher volume of liquid for the same core size. However, the addition of these polymers can reduce the fluid penetration time of the core 3. Due to its composition, the absorbent core tends to present low mechanical strength, especially when wet, whereby it is necessary to use a transfer layer 5, in an absorbent material, which also operates as a cohesion layer.

The transfer layer 5, which in one embodiment of the prior art is comprised of a tissue paper as mentioned above, provides a rapid and efficient absorption, conducting the body fluid from the discharge region to the absorbent core 3. Thus, the transfer layer 5 made of absorbent material provides rapid fluid transport.

Another function of the transfer layer 5, thereby also considered a cohesion layer, is to maintain the structural integrity of the absorbent core 3, even in case of a large quantity of moisture and the application of considerable mechanical forces thereon, such as for example, the compression caused by the wearer's legs. Thus, preferably, the transfer layer 5 envelops the absorbent core 3, cooperating to avoid disintegration thereof, which could interrupt the absorption capacity of the absorbent product 1 and result in fluid leakage. In the preferred embodiment, the transfer layer 5 envelops the absorbent core 3 so that, adjacent to the inner sheet 2, it presents a first overlapping region 51 onto which is placed a second overlapping region 52, configuring a double layer 53 made of absorbent material, i.e., a double transfer layer or even a double non-woven web, facing the inner side of the absorbent product 1 (the side that is going to receive the discharges of body exudates).

Thus, the absorbent product has the inner sheet 2 plus the two overlapping regions 51, 52 of the transfer layer 5 acting as elements with high fluid absorption and rapid fluid transport that function to rapidly conduct said fluids to the absorbent core 3 where they are stored. However, this embodiment provided with two overlapping regions 51, 52 is merely optional.

The transfer layer 5 consists of a non-woven web or structure made of polymeric material, being preferably polypropylene (100%), polyester (100%) or any other suitable material. Such web made of any polymeric s material is preferably produced by the spunbonding manufacturing process (formation via continuous spinning) and presents a basis weight substantially between 8 and 20 g/m², with the preferred values ranging between 8 and 12 g/m². Preferably, the web comprises low Denier fibers, of about 1.7D.

In order to guarantee the desired absorption capacity and penetration time properties, the transfer layer should be treated with a permanent surfactant element, preferably of the LERTISAN HD-14/HD-20 type in quantities preferably between 0.5% and 1.2%, to allow the material to maintain its properties of rapid absorption and rapid incorporation of fluids into the absorbent core 3, even after successive exudate discharges. However, other surfactant elements which function as “permanent surfactants” may also be used. Preferably, the transfer layer is treated by spraying or kiss-coating the surfactant onto the transfer layer. Alternatively, the surfactant may be otherwise coated or printed onto the transfer layer or formulated into the polymer before or during its formation into fibers.

The advantages of the present invention in relation to the products already available in the market can be better evaluated considering the information below.

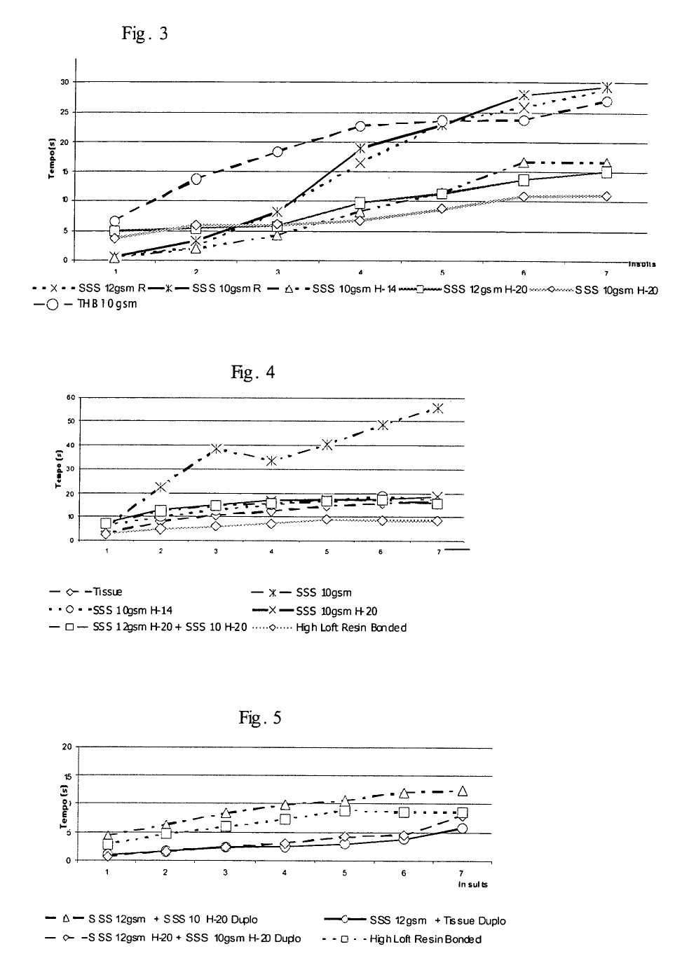

A test has been carried out, in which six inner sheets 2 of the present invention were evaluated, differing from each other as to basis weight, type and quantity of the surfactant used (traditional permanent LERTISAN HD-14/HD-20). The shortest liquid penetration times were obtained for the sheets having a permanent surfactant, after seven successive discharges. It can be appreciated from FIG. 3 that non-woven webs treated with the traditional non-permanent surfactant show a substantial and constant increase in the liquid penetration time after the different fluid discharges. For example, the SSS logsm R sample, a 10 gsm spunbonded polypropylene nonwoven (commercially available from Companhia Providencia, Parana, Brazil), shows a penetration time of 27 seconds on the seventh insult. In addition, the SSS 12 gsm R sample, a 12 gsm spunbonded polypropylene nonwoven (commercially available from Companhia Providencia, Parana, Brazil), shows a penetration time of 29 seconds on the seventh insult.

The penetration times of the known absorbent products, as compared to the absorbent product of the present invention, also varied. Non-woven sheets and webs with different surfactants as an insert were compared with tissue paper and a high loft resin bonded web. The results show that, in order to achieve results comparable to those obtained with tissue paper, it is necessary that the non-woven web used as transfer layer be treated with a permanent surfactant, so as to maintain the liquid penetration times at a low level. The system that uses high loft resin bonded web presents the best results. The results can be seen more precisely in FIG. 4.

In relation to the liquid penetration time of the absorbent structures using two layers of non-woven web as a transfer layer, it is possible to get results similar to those obtained with the known system using tissue paper and high loft resin bonded web, as seen in FIG. 5.

The test demonstrates that the use of two layers of a non-woven web with a low basis weight and treated with a permanent surfactant can satisfactorily substitute for the tissue paper, without interfering in the liquid penetration time.

The methodology of the test is described below:

Liquid Penetration Time

By using the “Strike Thru Tester” apparatus according to EDANA Rule 151.2:1999 (Brazilian Rule NBR 14796:2002), the liquid penetration time was determined after seven consecutive discharges of 5 ml each (saline solution 1%). For collecting the discharges, four layers made of cellulose pulp sheet of 680-700 g/m² were used under the non-woven webs to be evaluated.

Evaluations carried out in disposable diapers for babies, using a double non-woven web (object of the invention), as previously described, showed better values for the liquid penetration time and for the capacity of incorporating the liquid into the absorbent product (known as impact capacity).

BABY DIAPER

| | | DIAPER | | DIAPER |
|---|---|---|---|---|
| Medium Size | DIAPER A | B | DIAPER C | D |
| Penetration Time (s) | | | | |
| 1st penetration | 24.2 | 24.3 | 22.1 | 20.2 |
| 2nd penetration | 30.0 | 33.8 | 38.7 | 25.0 |
| 3rd penetration | 48.6 | 44.0 | 53.2 | 30.0 |
| Impact Capacity (g) | | | | |
| 1st penetration | 0.2 | 1.0 | 0.2 | 0.0 |
| 2nd penetration | 9.0 | 19.3 | 0.3 | 0.0 |
| 3rd penetration | 19.5 | 20.0 | 4.5 | 0.2 |
| Total | 29.6 | 40.3 | 5.0 | 0.2 |
| % | 39.5 | 53.7 | 6.7 | 0.4 |

DIAPER A: (Commercially available from Kimberly-Clark sold in Brazil under the trade name of TURMA DA MONICA) having an inner sheet of 15 grams/m² thermobonded polypropylene with a layer of 12-15 grams/m² thermobonded polypropylene nonwoven and a layer of 19-20 grams/m² Tissue Paper.

DIAPER B: (Commercially available from Procter & Gamble in Brazil under the trade name of PAMPERS NOTURNA) having an inner sheet (with DERMACREAM) of 13-17 grams/m² thermobonded polypropylene nonwoven with a layer of 40-50 grams/m² high loft resin bonded nonwoven and a layer of 19-20 grams/m² Tissue Paper.

DIAPER C: having an inner sheet of 12 grams/m² spunbond polypropylene with 0.8% to 1.0% LERTISAN HD-14/HD-20 with double layers of 19 grams/m² tissue paper.

DIAPER D: (An embodiment of the present invention) having an inner sheet of 12 grams/m² spunbond polypropylene with 0.8%-1.0% of LERTISAN HD-20 and double layers of 10 grams/m² spunbond polypropylene with 0.6%-0.8% of LERTISAN HD-20.

Liquid Penetration Time: measures the time (in seconds) that the absorbent product 1 takes to absorb three discharges of 80 ml of 0.9% saline solution in intervals of 10 minutes. The test method is according to the technical application test TA T-004 BASF of Brazil (method MA 0003-1 BASF Corporation).

Impact Capacity: measures the capacity of the absorbent product 1 to incorporate three discharges of 25 ml of 0.9% saline solution in intervals of 10 minutes, in an inclined plane of 30°. The test method is according to the technical application test TA T-007 BASF of Brazil (method MA 008-1 BASF Corporation).

As advantages, the absorbent product 1 of the present invention has a higher absorption capacity and a lower liquid penetration time, even after successive discharges, continuing to quickly absorb and conduct the body fluids from the discharge region to the absorbent core 3, allowing said fluids to be correctly stored therein. Moreover, the absorption time is even shorter than in all the other products, enhancing these advantages.

Also, the transfer layer 5 thus configured offers high mechanical strength, reducing the occurrence of structural collapse of the absorbent core 3 due to moisture combined with the application of forces. Furthermore, all these advantages are achieved, while maintaining a reduced manufacturing cost for the absorbent product 1, thus maximizing its potential of penetrating in the consumer market.

While a preferred exemplary embodiment has been described, it should be understood that the scope of the present invention encompasses other possible variations and it is only limited by the content of the appended claims and possible equivalents thereof.

1. An absorbent product, particularly for the absorption of body exudates, comprising an inner sheet to be positioned close to the wearer's body, an outer sheet to be positioned close to the wearer's garments, and an absorbent core positioned between said outer and inner sheets, the absorbent core being enveloped by a transfer layer made of an absorbent material, wherein the transfer layer comprises a polymeric material and presents a basis weight substantially between 8 and 20 g/m², and comprises, additionally, at least one permanent surfactant element.

2. The absorbent product, as set forth in claim 1, wherein at least adjacent to the inner sheet the transfer layer presents a first overlapping region onto which is provided a second overlapping region.

3. The absorbent product, as set forth in claim 2, wherein the overlapping of the first and second overlapping regions configures a double layer of absorbent material.

4. The absorbent product, as set forth in claim 1, wherein the transfer layer consists of polypropylene.

5. The absorbent product, as set forth in claim 1, wherein the transfer layer presents a basis weight substantially between 8 and 12 g/m².

6. The absorbent product, as set forth in claim 1, further comprising a permanent surfactant element in a range substantially between 0.2% and 1.5% of the weight of the transfer layer.

7. The absorbent product, as set forth in claim 6, further comprising a permanent surfactant element in a range substantially between 0.5% and 1.2% of the weight of the transfer layer.

8. The absorbent product, as set forth in claim 6, wherein the permanent surfactant element is present in a range substantially between 0.4% and 0.8% of the weight of the transfer layer.
